(19) United States
(12) Patent Application Publication
(10) Pub. No.: US 2022/0143186 A1
(43) Pub. Date: May 12, 2022

(54) SOLIDIFICATION OR CRYSTALLISATION METHOD

(71) Applicant: THE UNIVERSITY OF BRISTOL, Bristol (GB)

(72) Inventors: Simon Robert HALL, Bristol (GB); Jason Leigh POTTICARY, Bristol (GB); Charlie Lewis HALL, Bristol (GB)

(21) Appl. No.: 17/432,068

(22) PCT Filed: Feb. 19, 2020
(86) PCT No.: PCT/GB2020/050398

§ 371 Date: Aug. 18, 2021

(30) Foreign Application Priority Data
Feb. 19, 2019 (GB) 1902284.7

(51) Int. Cl. A61K 47/10; A61K 31/55; A61K 31/167; A61K 31/192; A61K 31/4985; A61K 31/421; A61K 31/166; A61K 31/405; A61K 31/53; A61K 31/437

(52) U.S. Cl. CPC A61K 47/10; A61K 31/55; A61K 31/167; A61K 31/192; A61K 31/437; A61K 31/421; A61K 31/166; A61K 31/405; A61K 31/53; A61K 31/4985

(57) ABSTRACT

A solidification or crystallization method is disclosed, which includes providing at least a first organic compound and at least one volatile co-former organic compound. A mixture of at least the first organic compound and the co-former organic compound is formed, wherein either the first organic compound or the volatile co-former organic compound includes a hydrogen acceptor moiety and the other includes a hydrogen donor moiety, thereby allowing the formation of hydrogen bonds between the first organic compound and the volatile co-former organic compound. The mixture is allowed to stand for sufficient time for the mixture to liquify at a temperature below that of the melting points of the components, thereby forming a liquid mixture. The volatile co-former organic compound is allowed to evaporate, thereby resulting in crystallization of at least the first organic compound. The method can be a co-crystallization method if there are two organic compounds.

### FIELD

The present disclosure relates to solidification, preferably crystallization methods. More particularly, the present disclosure relates to solidification/crystallization methods for active ingredient systems and co-solidification/co-crystallization methods for two- or multi-component systems. The present disclosure also relates to eutectic mixtures, glasses and co-crystalline or amorphous solids of active ingredients.

### INTRODUCTION

Deep eutectic solvents (DES) are known and have been investigated over the last two decades (see for example, Q. Zhang, et al; Chem. Soc. Rev. 41 (2012), pp. 7108-7146).

DES systems are composed of two or three molecular species which are able to associate through extensive hydrogen bonding. An appropriate choice of two components (usually solid and crystalline) forms a liquid with a melting point significantly lower than each of the constituents.

One of the first examples of a DES was formed by the mixture of solid choline chloride and urea in a molar ratio of 1:2. This eutectic mixture is a liquid at 12° C., whilst the component parts have melting points of 302° C. and 133° C. respectively. (A. P. Abbott, et al.; Chem. Commun. 9, 70-71 (2003)). Other DES systems have been formed by mixing a quaternary ammonium salt with a hydrogen bond donor (HBD). DES systems have been used in studies relating to catalysis, extraction processes, electrochemistry, organic synthesis, batteries and dye-sensitized solar cells. DES are being currently investigated especially in the pharmaceutical industry to address problems with compound solubility, particularly of some poorly soluble polymorphs of active pharmaceutical ingredient since up to 90% of new chemical entities considered for bringing to market are classed as poorly soluble (GlobalData Healthcare, CPHI experts: Pharmaceutical Technology; available at https://www.pharmaceutical-technology.com/comment/cphi-experts-90-current-pipeline-apis-poorly-soluble/).

When developing an active pharmaceutical ingredient (API), an important consideration is bio-availability. A change in polymorphic form can result in changes to intermolecular interactions and the crystal surface chemistry which can adversely affect solubility, dissolution rate and intestinal permeability. In addition, different polymorphs of an active pharmaceutical ingredient (an “API”) often have different crystal habits which can have a significant impact on the processability of the API. For example, higher cohesion between crystals with higher areas of exposed polar surfaces may lead to clogging of processing hardware. Mechanical processes such as milling may be used to reduce particle size. However, milling may result in mechanically induced solid-state transformations of the API and agglomeration of the produced particles.

Generally, the most thermodynamically stable polymorph of an API is the least soluble when compared to higher energy metastable polymorphs. However, these higher energy polymorphs often require more difficult crystallization conditions or complex crystallization routes such as de-solvation or epitaxial growth. For example, paracetamol (acetaminophenol) has a number of polymorphs, two of which are stable under ambient conditions: forms I and II. Paracetamol is manufactured and distributed as form I, a less efficacious form, due to its ease of production and crystalline stability. Form II is more soluble and is more readily compressed into tablets, however it is more difficult to crystallize, requiring additives or higher temperature. Obtaining higher energy, more efficacious polymorphs of any API may not be a cost-effective strategy when having to scale up. There is a requirement therefore for a simple route to hard-to-reach or novel polymorphs which is scalable and works at or near room temperature and pressure.

There has been interest in materials comprising two compounds in cocrystalline form. Cocrystals are usually considered to consist of two or more components that form a unique crystalline structure having unique properties. Because of their unique properties, often different to the properties of their components, cocrystals are receiving interest as potentially improved active pharmaceutical ingredients, fertilizers, pesticides, foodstuffs, field-effect transistors, solid-state organic lasers, organic superconductors, pigments, explosives and detergents.

US-A-2005/0181041 A1 discloses methods of preparing an active agent as mixed phase co-crystals that have unique physical properties that differ from the active agent in pure form, as well as compositions comprising mixed phase co-crystals. The method uses a simple solvent/anti-solvent system, with solvents such as DMSO and anti-solvents such as water in which one component is crystallized in the presence of another, producing an admixture of the two active ingredients. The formulated mixed phase co-crystals are heterogenous and contain crystalline regions within the particles/granules produced.

CN-A-106 187 855 A discloses a method using a deep eutectic solvent of choline chloride and zinc chloride as a reaction medium for the synthesis of 2-arylindole compounds and requires a reaction between phenyl hydrazine and a substituted acetophenone in the liquid phase between 120 to 125° C.

There is a need to provide improved processes that can be used to produce compounds of varied morphology and crystal structure in both single and multicomponent systems.

### SUMMARY

It is an aim of the present disclosure to address this need.

In a first aspect, the present disclosure accordingly provides a solidification method preferably a crystallization method, the method comprising:

providing at least a first organic compound,

providing at least one volatile co-former organic compound,

forming a mixture of at least the first organic compound and the co-former organic compound, wherein either the first organic compound or the volatile co-former organic compound comprises a hydrogen acceptor moiety and the other comprises a hydrogen donor moiety, thereby allowing the formation of hydrogen bonds between the first organic compound and the volatile co-former organic compound,

allowing the mixture to stand for sufficient time for the mixture to liquify at a temperature below that of the melting points of the components, thereby forming a liquid mixture, and

allowing the volatile co-former organic compound to evaporate, thereby resulting in crystallization of at least the first organic compound.

These mixtures, where the crystals form from deep eutectic systems containing a relatively volatile co-former compound are referred to in this specification as deep eutomic solvents (DXS).

This is greatly advantageous because surprisingly such methods allow more control over crystal polymorph and morphology and, where there is a second organic compound, the formation of co-solidified solids, preferably co-crystalline solids.

Methods of the present disclosure may be used to form eutectic mixtures.

Thus, in a second aspect, the present disclosure provides a eutectic mixture comprising phenol and a first organic compound selected from carbamazepine, paracetamol, metacetamol, ibuprofen, tadalafil, metaxalone, benzamide, 2-methoxybenzamide, 2-ethoxybenzamide, indomethacin, lamotrigine and harmine and/or two or more of these compounds.

Preferably, the eutectic mixture is liquid at room temperature and pressure.

Usually, phenol and the first organic compound are in a molar ratio phenol:first organic compound in the range 10:1 to 2:1.

In a third aspect, the present disclosure provides a eutectic mixture comprising benzamide, metaxalone and phenol, which is liquid at room temperature and pressure.

Usually, benzamide, metaxalone and phenol are in a molar ratio benzamide:metaxalone:phenol in the range 0.1:1.9:10 to 1.9:0.1:10.

In a fourth aspect, the present disclosure provides a eutectic mixture comprising metaxalone, carbamazepine and phenol, which is liquid at room temperature and pressure.

Usually, carbamazepine, metaxalone and phenol are in a molar ratio carbamazepine:metaxalone:phenol in the range 0.1:1.9:10 to 1.9:0.1:10.

After eutectic mixtures with more than one organic compound are formed according to the methods of the present disclosure, allowing the volatile hydrogen bond donor compound to evaporate (with or without heating or reduced pressure) results in formation of cocrystals.

Thus, in a fifth aspect, the present disclosure provides a co-crystalline solid comprising benzamide and metaxalone, optionally in a molecular ratio in the range 2:1 to 1:2.

In a sixth aspect, the present disclosure provides a co-crystalline solid comprising metaxalone and carbamazepine, optionally in a molecular ratio in the range 2:1 to 1:2.

In further aspects, the present disclosure provides a co-crystalline solid comprising 2′-Aminoacetanilide and tetracyanoquinodimethane (TCNQ) or a co-crystalline solid comprising theobromine and vanillic acid.

Products from the methods of the present disclosure may be pharmaceuticals (having improved bio-availability, processing ability or effect), fertilizers and pesticides (e.g. with slow dissolution and release), foodstuffs (e.g. longer shelf-life ingredients), field-effect transistors (e.g. with higher conductivity), improved solid-state organic lasers, organic superconductors (e.g. with higher superconducting critical temperature), pigments (e.g. with longer colorfastness), explosives (that may be less shock sensitive) or improved detergents.

### BRIEF DESCRIPTION OF THE DRAWINGS

FIG. 1 is a graph showing the solubility of paracetamol in different organic solvents. The grey bar represents the solubilities achieved by the phenol DXS before decomposition.

FIG. 2 is a graph showing the crystalline form of PAP and MAP as a function of both time and HBD:HBA ratio. (a) Hatched and grey squares indicate the appearance of forms I and II of paracetamol, respectively. (b) Grey and hatched blocks indicate the appearance of dendrites and fibers of metacetamol, respectively. Days indicated on the y-axis denote time since the DXS was formed.

FIG. 3 shows H-bonding motifs in crystalline forms of PAP. (a) A chair-like cycle of interacting PAP molecules in form I. The dashed arrows and axes indicate the angles and sterically obstructed approach vectors. (b) The cycle of interacting PAP molecules in form II showing a more ‘planar’ structure. Individual molecules are colored for ease of visualization. Black lines numbered 1 and 2 indicate H-bonds of lengths (a) 1=2.049 Å and 2=1.796 Å and (b) 1=2.118 Å and 2=1.835 Å.

FIG. 4 shows H-bonding motifs in crystalline forms of MAP. (a) Upper: Helical chain and lower: linear chain of interacting MAP molecules in form I. (b) Dimer of interacting MAP molecules in form II. Individual molecules are shaded for ease of visualization. Black lines indicate the H-bonds bonds of lengths (a) 1=2.080 Å and 2=1.819 Å and (b) 1=1.849 Å and 2=1.837 Å.

FIG. 5 shows micrographs of different crystalline forms of harmine (Example 14). (a) Optical image of crystals of harmine phenolate and (b) an optical image of same crystals after vacuum drying to remove the phenol leaving the standard harmine crystal structure. (c) SEM micrograph of the surface of the dried harmine in (b) revealing a porous micromorphology with an average pore size of 0.22 μm.

FIG. 6 is an X-ray powder diffraction pattern of the product of Example 16 (carbamazepine).

FIG. 7 is an X-ray powder diffraction pattern of the product of Example 17 (metaxalone).

FIG. 8 is an X-ray powder diffraction pattern of the product of Example 18 (oxcarbazepine).

FIG. 9 is an X-ray powder diffraction pattern of the product of Example 19

(PAP).

FIG. 10 is an X-ray powder diffraction pattern of the product of Example 22 (urea/4-nitrophenol).

FIG. 11 is an X-ray powder diffraction pattern of the product of Example 23 (p-coumaric acid/nicotinamide).

FIG. 12 is an X-ray powder diffraction pattern of the product of Example 24 (4-hydroxybenzoic acid/tebuconazole).

FIG. 13 is an X-ray powder diffraction pattern of the product of Example 11 (2-Ethoxybenzamide).

FIG. 14 is an X-ray powder diffraction pattern of the non-phenolated product of Example 14 (Harmine).

FIG. 15 is an X-ray powder diffraction pattern of the phenolate product of Example 14 (harmine).

FIG. 16 is an X-ray powder diffraction pattern of the product of Example 13

(Lamotrigine).

FIG. 17 is an X-ray powder diffraction pattern of the product of Example 5 (Metacetamol).

FIG. 18 is an X-ray powder diffraction pattern of the product of Example 20 (Benzamide/Metaxalone).

FIG. 19 is an X-ray powder diffraction pattern of the product of Example 21 (Metaxalone/Carbamazepine).

FIG. 20 is an X-ray powder diffraction pattern of the product of Example 15 (Vemurafenib).

FIG. 21 is an X-ray powder diffraction patterns of the product of Example 25 (2′-Aminoacetanilide and tetracyanoquinodimethane, TCNQ).

FIG. 22 is an X-ray diffraction pattern of the product of Example 26 (theobromine and vanillic acid).

### DETAILED DESCRIPTION

The present disclosure is further illustrated, but not limited, by the following examples.

Powder X-ray diffraction (pXRD) data were gathered using a Bruker D8 Advance diffractometer (Cu-Kα radiation—wavelength of 1.5418 Å) with a PSD LynxEye Detector.

Samples for NMR were prepared by dissolving 50 mg of sample in 0.7 cm³ of deuterated solvent with a tetramethylsilane reference standard and filtered. All NMR measurements were carried out on a Jeol ECS-400.

In the Examples, reference is made to deep eutomic solvents (DXS), which as discussed above are deep eutectic systems containing a relatively volatile hydrogen bond donor or acceptor compound as co-former

The DXS systems may comprise a volatile hydrogen bond donor compound (HBD) and one or more hydrogen bond acceptor compounds (HBA). In each of Examples 1 to 14, there is one HBA.

In some aspects, therefore, a DXS may comprise a volatile HBD and stable HBA component (e.g. in the ratios 1:1-10:1—HBD:HBA, respectively) which, when simply mixed together as solids, produces a liquid which remains stable in a sealed container at or near room temperature. This admixture may subsequently be left to ‘self-destruct’ at room temperature and pressure for a time, Tx (typically ˜36 hr), resulting in the spontaneous crystallization of the non-volatile component. A pharmaceutical compound may be used as the HBA component, which means that in lieu of dissolution of and concentrations in a solvent, the liquid produced, is itself, part API; in some cases the API comprises 20% of the liquid.

In Examples 1 to 14, the components were in ratios 1:1 to 10:1 (HBD:HBA), which produced a liquid which remained stable in a sealed container at room temperature. Once the liquid was homogeneous, droplets of the DXS left under ambient conditions allowed the HBD to evaporate resulting in spontaneous crystallization of the HBA. The range of ratios at which a stable DXS is formed affords an easily tunable range of concentrations with regards to the API in the solvent. All eutomic mixtures formed exhibited deep eutectic behavior in that there was melting point depression or glass transitions temperature depression, with the melting point or glass transition points significantly lower in temperature than those of the components (Table 1).

### Example 1 Morphology of Benzamide Crystals

Example 1 relates to a DXS system consisting of phenol as the HBD and benzamide as the HBA. Benzamide is a good model system for an API as it has a structural motif found in many drugs and has three known forms (forms I, II and III).

The highly metastable form II and form III are formed concomitantly at higher supersaturations but when dissolved in benzene will transform to form I over time.

The effect of altering functional groups on the eutomic behavior was examined using 2-methoxybenzamide (2MB) and 2-ethoxybenzamide (2EB), as simple variations on the underlying benzamide structure

Phenol:benzamide mixtures were prepared with molar ratios in the range 4:1 to 9:1, all of which resulted in a homogeneous clear liquid. On allowing phenol to evaporate, large crystals of the form III polymorph were produced, interspersed with opaque needles of form I. Time-lapse imagery of the formation of these needles suggests that a metastable crystal is forming, followed by the rapid conversion to form I. Upon aging in quiescent storage prior to HBD evaporation however, a DXS of ratio 9:1 phenol:benzamide consistently gave only the form III polymorph. The lack of conversion from form III to form I from the aged solutions is suggestive that no form I is present at any point during the crystallization. We can understand this mechanistically through consideration of the molecular interactions between the HBD and HBA. The capacity to form intermolecular hydrogen-bonding networks have been shown to be advantageous in DES formation, indeed there must be a propensity for the HBD:HBA interaction to be energetically more favourable than molecular self-interaction. We have found that the introduction of steric effects in benzamide derivatives alter the tendency for DXS formation and destruction. These altered interactions can be correlated with a change in melting point as a function of increasing steric interactions of the HBD and HBA. The lowest recorded melting points of benzamide, 2MB and 2EB eutectics are found to be −37.57° C., −38.87° C. and −37.91° C. respectively, which is also reflected in the speed at which these mixtures form liquids when left to stand together. Interestingly 2MB is the most freely forming DXS with the lowest melting point implying that a balance between number and accessibility of hydrogen bonding sites is advantageous when designing a DXS system.

### Example 2: Polymorphs of Acetaminophen

Isomers of acetaminophen, where the acetamide group can be on three possible ring positions, para-, and meta- (PAP and MAP respectively) were investigated.

PAP has two common polymorphs, form I, is based around a catemeric arrangement and crystallizes from organic solvents whereas form II is grown from the melt and is based on a stacked dimer. A third isomer, OAP is the least studied, with no reported crystal structures or powder patterns. OAP currently has no current industrial or pharmaceutical applications.

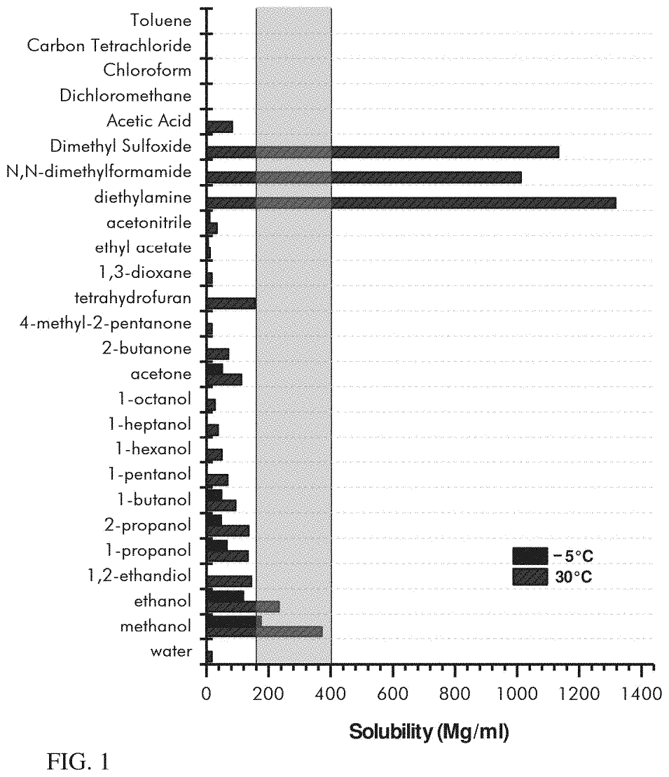

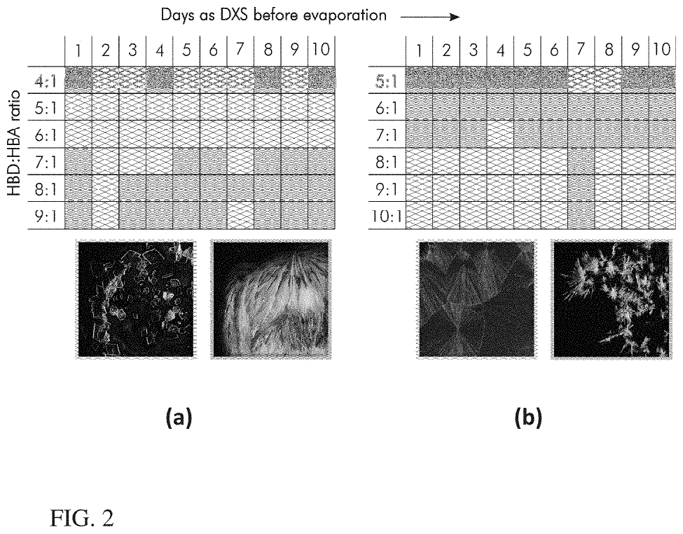

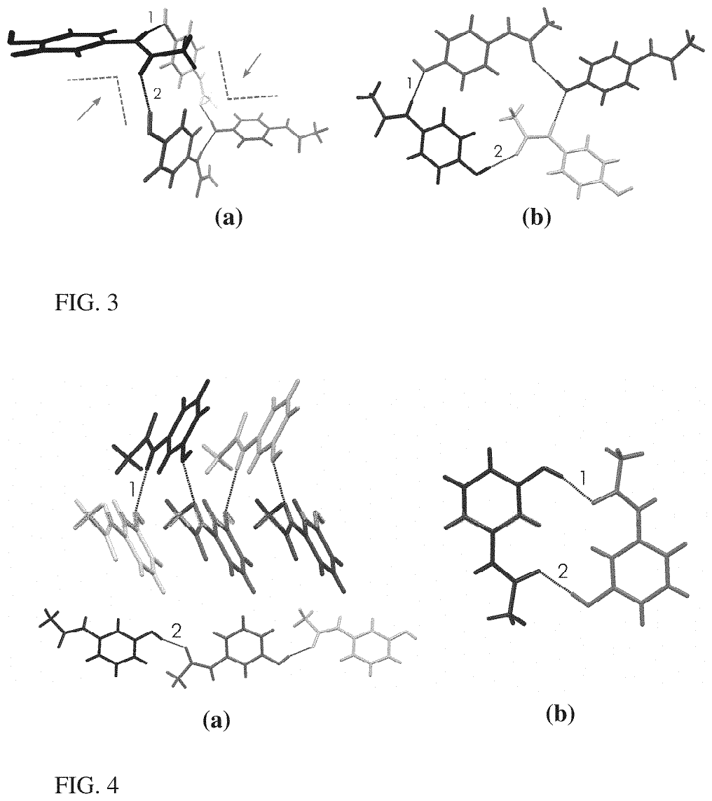

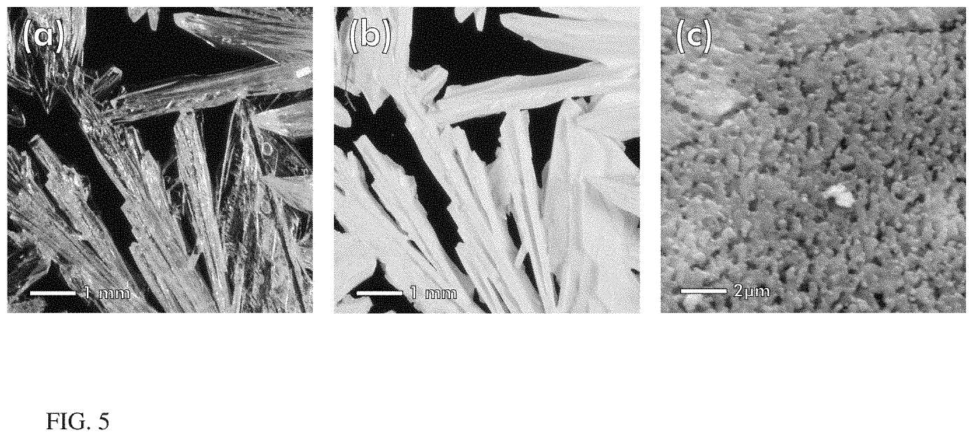

As with the benzamide system, samples of PAP, and MAP (as HBD) were mixed with phenol to form a homogeneous liquid DXS. PAP and MAP were found to produce a stable DXS between the molar ratios of 4:1 and 9:1 and 5:1 and 10:1 (HBD:HBA), respectively. When in the para position there are a wider range of angles available for the phenol to approach and H-bond with the API, enabling the formation of a stable DXS at lower HBD:HBA ratios. The DXS systems created from PAP and MAP are so easily formed that they allow crystals to be grown from a solution with API concentrations not achieved using many common organic solvents (see FIG. 1). In the case of PAP, the most efficacious polymorph (form II) currently considered commercially unsuitable for production due to difficulties discussed above, will emerge from a DXS spontaneously at room temperature and pressure, without the addition of any additives, templates or epitaxial constraints. Evaporation of the volatile phenol component leads to the formation of PAP polymorphs over a 10-day period. Analysis of resultant crystals from phenol:PAP ratios from 4:1-9:1 show a change from one morphology to the other, usually between the ratios 6:1 and 7:1. Powder X-ray diffraction analysis shows that this difference is due to a different polymorph being formed, namely form I (4:1-6:1) and form II (7:1-9:1) of which the crystal habit observed is characteristic; diamonds in the case of form I and needles in form II (see FIG. 2). As can be seen, with a ratio of 4:1 phenol:PAP, a mixture of polymorphs is observed although most commonly the PAP polymorph that crystallizes is form I. When the ratio is increased to 5:1 and 6:1, exclusively form I is observed over the course of ten days, in contrast to ratios of 7:1-9:1, which are dominated by form II. MAP shows different crystalline morphologies to that seen in PAP because only a single polymorph, form I, is observed.

Both forms I and II of PAP contain a hydrogen-bonded ring of four molecules as a structural sub-unit. The structure of the ring in form I contains molecules orthogonally disposed to each other (FIG. 4(a)), whereas molecules in form II sit almost parallel to each other (FIG. 4(b)). Results from the calculations show that the form I motif has fewer sites for phenol molecules to occupy than in the form II motif, however, they are at lower energies. The result of this is that the addition of extra phenol molecules will promote the formation of form II hydrogen-bonding motifs and the therefore the appearance of the polymorph change at high phenol concentrations.

### Examples 3 to 15: Other DXS Systems with APIs as Hydrogen Bond Acceptors

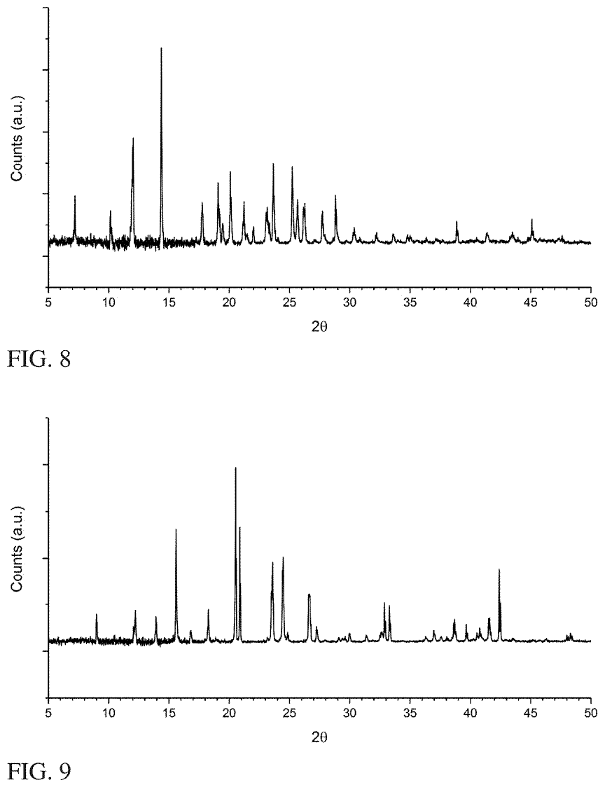

Table 1 describes a list of other APIs used as hydrogen-bond acceptors. In some cases, formation of a stable cocrystal of API and phenol occurs, with the phenol effectively playing the role of ‘solvate’ in the crystal. These solvate structures, lead to known forms of the API upon evaporation of phenol and de-solvation of the crystal. In the case of harmine, a reversible monoamine oxidase inhibitor, the phenolate is a precursor to the only know native crystalline form. However, upon gentle heating or vacuum drying of this phenolate, the macro-morphology of the crystal is preserved (FIG. 8(b)), but the large single-crystals (FIG. 8 (a)) have been transformed into a porous, polycrystalline matrix through loss of phenol (FIG. 8(c)). This suggests another use of DXS systems may be the production of high surface area, high dissolution rate APIs.

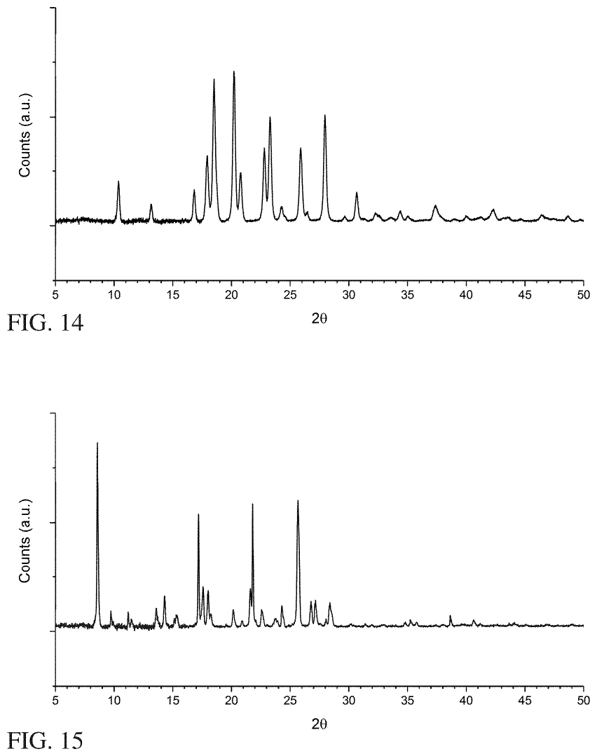

As well as harmine, 2EB has been found to have a stable phenol cocrystal observable during phenol evolution. Both of these structures are 1:1 HBD:HBA and have been solved (CCDC deposit numbers 1879689 and 1879336, respectively). Although these cocrystals are stable enough to withstand structure determination, formation and subsequent de-solvation are likely the key drivers of the complex thermal behavior observed via DSC. Interactions between components in a DES are dynamic and are facilitated by an array of possible bonding motifs. For example, the start of crystallization of a 1:1 cocrystal changes the concentrations of the liquids in the system which may lead to a change in the nature and number of molecular interactions throughout the system. X-ray diffraction results for harmine and the harmine phenolate products are shown in FIGS. 14 and 15 respectively.

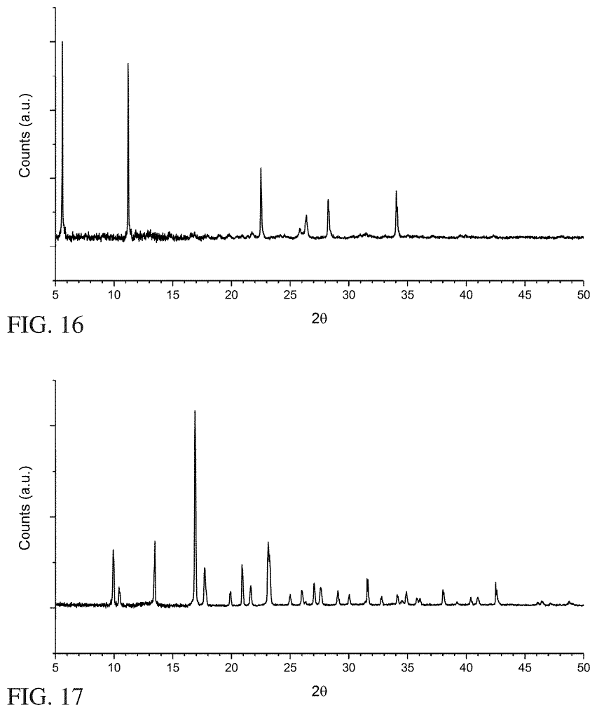

The results of X-ray diffraction study of the product of Example 11 (2-ethoxybenzamide) is shown in FIG. 13. The results of X-ray diffraction of the products of Example 13 (lamotrigine) is shown in FIG. 16, and of Example 5 (metacetamol) in FIG. 17.

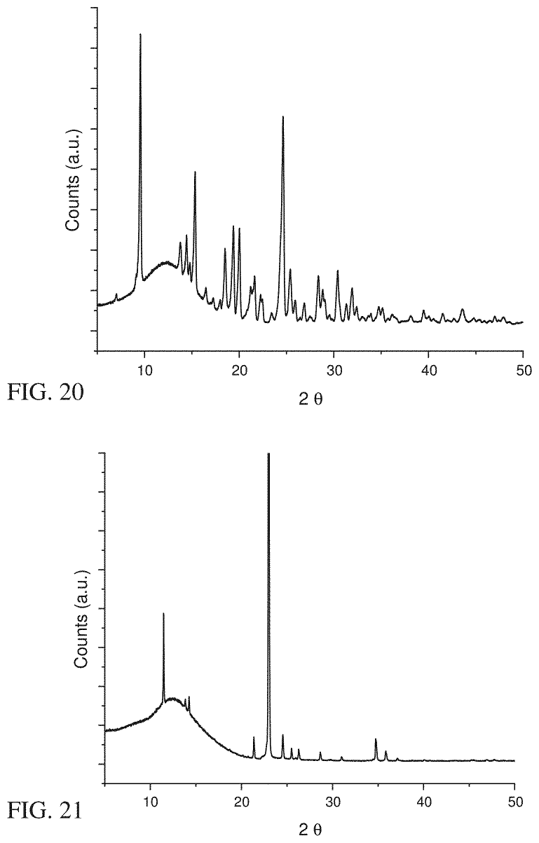

In Example 15, the API Vemurafenib, insoluble in only nanograms/ml in most organic solvents, was mixed with phenol as an HBD in a ratio of 10:1 to form a stable liquid. The HBD was left to leave the system, generating crystals of the API. The results of X-ray powder diffraction of the product of Example 15 are shown in FIG. 20.

A number of similar studies were conducted and some of the studies above repeated with the results set out in Table 2, below. In these repeated studies, once completely liquid, droplets of the DXS were left under ambient conditions for the HBD to evaporate resulting in destruction of the DXS and spontaneous crystallization of the pharmaceutical HBA. Subsequent NMR of solutions of the as-crystallized HBA showed no detectable residual HBD present. The range of ratios at which a stable DXS is formed affords an easily tunable array of concentrations with regards to the API in the solvent and it is a feature of the DXS system that eutomic mixtures formed usually exhibited deep eutectic behavior with melting point depressions or glass transitions significantly lower than the component parts ranging from ˜29° C. to sub −70° C. (Table 2).

TABLE 1
Summary of API DES properties, HBD is phenol for each example

| | | | | HBA Mₚ^{†} | Lowest recorded | Polymorph | Crystal habit |
|---|---|---|---|---|---|---|---|
| Example | API | Min^{*} | Max^{*} | (° C.) | Mₚ (° C.) | selectivity? | selectivity? |
| 3 | Carbamazepine | 2:1 | 10:1 | 192 | <−70 | Y | N |
| 4 | Paracetamol | 4:1 | 10:1 | 169 | −32.58 | Y | N |
| 5 | Metacetamol | 2:1 | 10:1 | 147 | −15.05 | N | Y |
| 6 | Ibuprofen | 1:1 | 10:1 | 76 | −23.98 | N | Y |
| 7 | Tadalafil | 6:1 | 10:1 | 301 | −20.99 | N | Y |
| 8 | Metaxalone | 3:1 | 10:1 | 122 | −31.84 | Y | N |
| 9 | Benzamide | 4:1 | 10:1 | 127 | −37.57 | Y | N |
| 10 | 2-Methoxybenzamide | 4:1 | 10:1 | 127 | −38.87 | N | N |
| 11 | 2-Ethoxybenzamide | 4:1 | 10:1 | 132 | −37.91 | N | N |
| 12 | Indomethacin | 3:1 | 10:1 | 311 | −22.74 | N | N |
| 13 | Lamotrigine | 5:1 | 10:1 | 216 | 22.34 | Y | N |
| 14 | Harmine | 4:1 | 10:1 | 321 | 32.42 | N | Y |

^{*}Described as HBD:API ratio
^{†}Of the common polymorph
Eₚ-Ratio of eutectic point

TABLE 2
Summary of melting points of other studies for some of the compounds considered
in this study and that of their respective DXS.

| | Designation in | | | Lowest m.p. |
|---|---|---|---|---|
| Compound | this study | HBD | Lowest DXS | ratio |
| (m.p. (° C.)) | (see Table 3) | (m.p. (° C.)) | m.p./T_{g} (° C.) | (HBD:HBA) |
| Phenol (40.5) | n/a (1) | — | — | — |
| Paracetamol (169) | PAP (2) | Phenol | <−70 | 7:1-10:1 |
| | | (40.5) | | |
| Metacetamol (149) | MAP (3) | Phenol | −58 | 4:1 |
| | | (40.5) | | |
| Benzamide (130) | n/a (5) | Phenol | −19 | 10:1 |
| | | (40.5) | | |
| 2-methoxybenzamide (128) | 2MB (6) | Phenol | <−70 | 4:1 |
| | | (40.5) | | |
| 2-ethoxybenzamide (134) | 2EB (7) | Phenol | <−70 | 4:1 |
| | | (40.5) | | |
| Carbamazepine (192) | n/a (8) | Phenol | <−64 | 9:1 |
| | | (40.5) | | |
| Harmine (321) | n/a (9) | Phenol | n/a | n/a |
| | | (40.5) | | |
| Metaxalone (122) | n/a (10) | Phenol | −58 | 4:1 |
| | | (40.5) | | |
| Verapamil hydrochloride (139) | n/a (11) | Phenol | <−70 | 10:1 |
| | | (40.5) | | |

TABLE 3
Formulae of selected compounds. Numbers of the formulae are indicated
in Table 2.

| | |
|---|---|
| 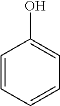 | 1 |
| 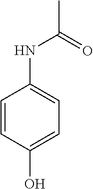 | 2 |
| 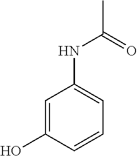 | 3 |
| 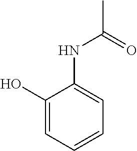 | 4 |
| 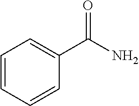 | 5 |
| 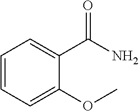 | 6 |
| 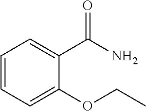 | 7 |
| 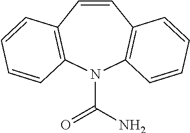 [SMILES: NC(=O)N1c2ccccc2C=Cc2ccccc21] | 8 |
| 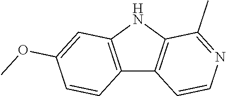 [SMILES: COc1ccc2c(c1)[nH]c1c(C)nccc12] | 9 |
| 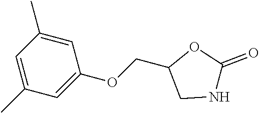 [SMILES: Cc1cc(C)cc(OCC2CNC(=O)O2)c1] | 10 |
| 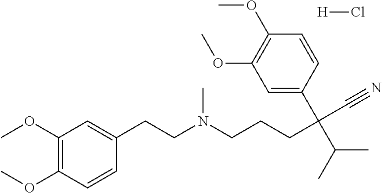 | 11 |

### Thermal Analyses of Deep Eutomics

To understand the extent of melting point depression, differential scanning calorimetry (DSC) analysis was performed on all compositions and ratios of API:phenol. DSC analysis was performed using a TA Instruments Q2000 with refrigerated cooling system. The DSC cell was purged with nitrogen. Samples were analyzed in hermetically sealed aluminum pans. A small volume of liquid (˜2-5 μL) was added to the hermetic pan and sealed. All liquid samples were cooled from 25° C. to −70° C. and then heated to 100° C. all at 10° C./min. The instrument was calibrated using a pure indium standard.

All DXS systems show large melting point depression for all molar ratios studied. Melting endotherms are broad and often occur at or just after the crystallization exotherm leading to a ±5° C. uncertainty in melting point depression. Melting points in the region 0-11° C. are observed for all compositions studied of paracetamol and phenol (c.f. paracetamol Form I with Tₘ˜169° C.). For metacetamol (Tₘ˜147° C.), melting point depression is not as marked as for paracetamol, with melting points in the range 8-30° C. Benzamide (Tₘ˜127° C.), 2-methoxybenzamide (Tₘ˜127° C.), 2-ethoxybenzamide (Tₘ˜132° C.), two other APIs, Metaxalone (Tₘ 122° C.), and Carbamazepine (Tₘ˜192° C.) phenol DES's show melting onsets in the ranges −6 to 15° C., −19° C. to 1° C., −9° C. to 22° C., −3 to 13° C., and −10 to −1° C. respectively. Although the melting point depression within a particular system is consistent within a narrow temperature range, there are some differences in physical behavior. For instance, metacetamol:phenol 1:3 does not crystallize upon cooling from ambient to −70° C. but will crystallize upon heating at ˜−17° C. followed by a melt at −2° C. For metacetamol:phenol 1:5, a crystallization event is observed upon cooling from ambient at ˜−13° C. Melting is subsequently observed upon heating at ˜25° C. (see supplementary information).

Suppression of the melting point in the optimum deep eutomic ratio leads to a crystallization temperature often 40° C. below room temperature (60° C. below that of phenol) and usually greater than 100° C. below room temperature. It is clear from our modelling of these systems that the mechanisms of crystallization from a deep eutomic solution are related to both the molecular structure of the API and the packing within the resultant crystals. The propensity for potential hydrogen bonding with a co-former appears to be advantageous in determining how likely an API is to form a DXS and at what ratios before there is crystallization of either the API at one extreme or the co-former at the other. This is exemplified in the case of PAP, MAP and OAP; as the available locations for hydrogen-bond interactions decreases, so do the concentrations of API in the solvent. In the benzamide system, spontaneous formation of a stable liquid is fastest with 2-methoxybenzamide, where there are both a carboxamide and an ester moiety present, in contrast to benzamide and 2-ethoxybenzamide, whose ester is more sterically hindered.

### Examples 16 to 19: DXS Systems with the Volatile Co-Former being a Hydrogen Bond Acceptor

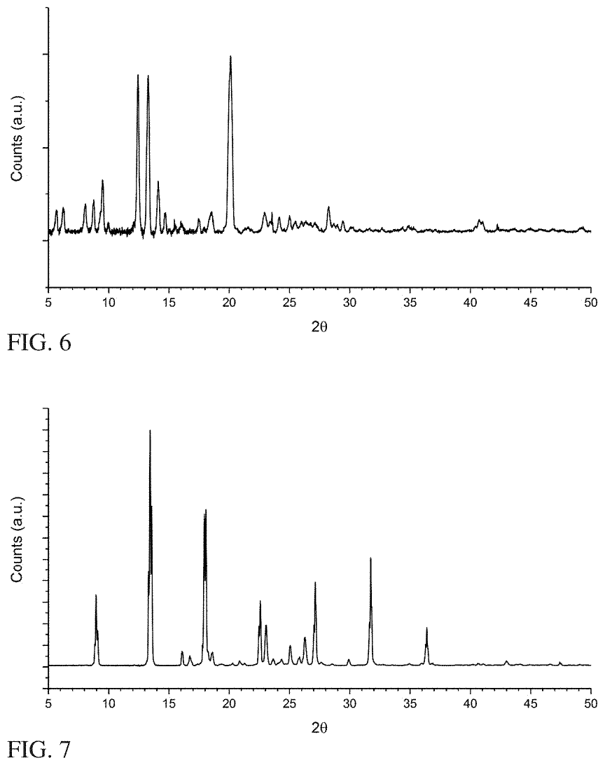

In Example 16, volatile acetophenone and carbamazepine were used to form crystalline carbamazepine, using a method as set out above in Example 3 to 15 with, in this case the volatile co-former organic compound being a hydrogen bond acceptor (acetophenone) and carbamazepine a hydrogen bond donor. An X-ray diffraction powder pattern for the resulting crystalline solid is shown in FIG. 6.

In Example 17, volatile acetophenone and metaxalone were used to form crystalline metaxalone, using generally the same method with the volatile co-former organic compound being a hydrogen bond acceptor (acetophenone) and metaxalone a hydrogen bond donor. An X-ray diffraction powder pattern for the resulting crystalline solid is shown in FIG. 7.

In Example 18, volatile acetophenone and oxcarbazepine were used to form crystalline oxcarbazepine, using generally the same method with the volatile co-former organic compound being a hydrogen bond acceptor (acetophenone) and oxcarbazepine a hydrogen bond donor. An X-ray diffraction powder pattern for the resulting crystalline solid is shown in FIG. 8.

In Example 19, volatile acetophenone and PAP were used to form crystalline PAP, using generally the same method with the volatile co-former organic compound being a hydrogen bond acceptor (acetophenone) and PAP a hydrogen bond donor. An X-ray diffraction powder pattern for the resulting crystalline solid is shown in FIG. 9.

### Examples 20 to 26: Formation of Co-Crystals

Co-crystals have been produced using a mixture of two organic compounds and a hydrogen bond donor that is volatile at room temperature and pressure. The organic compounds may be active pharmaceutical ingredients, fertilizers, pesticides, foodstuffs, field-effect transistors, solid-state organic lasers, organic superconductors, pigments, explosives or detergents. The volatile hydrogen bond donor may be, for example, phenol, hydroquinone, resorcinol, catechol or cyclohexanol, or can be a hydroxy-functionalized aromatic compound that is volatile at room temperature and pressure. Depending on the composition, the melting point of the deep eutectic mixture of the volatile and non-volatile components can be considerably lower than the melting point of any of the individual components.

Embodiments of the present disclosure comprise a eutectic solvent having two non-volatile organic compounds and a hydrogen bond donor volatile at room temperature and pressure. This allows, on exposure to air, the hydrogen bond donor to evaporate from the deep eutectic system and thereby induce the other two non-volatile organic molecules to co-crystallize, either as a fully crystalline form or as a homogeneous amorphous glass.

Depending on the composition, the melting point of the mixture may be significantly lower than the melting point of any of the three components.

### Experimental Method

### Preparation of Deep Eutectic Solvents

A stoichiometric molar ratio of a first and a second organic compound is weighed out and mixed together (in a molar ratio of 1:1). Once mixed, a molar amount of the volatile hydrogen bond donor compound co-former is added at a ratio found to provide a liquid at room temperature (molar ratio 1:1:10). This mixture is sealed and left to stand at room temperature and pressure to liquify, occasionally mild heating (50° C.) is required to ensure the liquification of all solids. Once a stable liquid has formed, the vessel may be unsealed so that the volatile co-former may evaporate which results in formation of a co-crystalline solid comprising the first and second organic compounds.

In order to determine the melting point of the two specific examples of a deep eutectic solution and to ensure that the DES in each case was a deep eutectic solvent, the mixture was cooled in an air chiller to below 0° C., whereupon the viscosity gradually increased, leading to a solidified glassy state without any crystallization. The solutions were cooled using a SP Scientific XR902 AirJet air chiller. The temperature was monitored using a Testo 174/175 temperature logger with a k-type thermocouple probe. Such a depression of melting point means that these DESs are able to be made and stored in liquid form under the majority of ambient temperatures This should contribute significantly to their ease of transportation and use.

### Example 20: Co-Crystalline Benzamide/Metaxalone

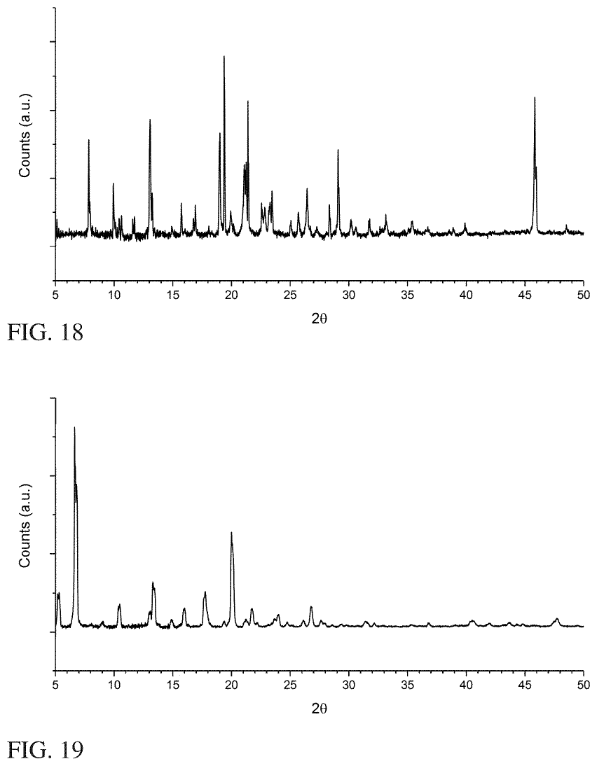

In Example 20, samples of the pharmaceuticals benzamide (melting point 130° C.) and metaxalone (melting point 122° C.), with phenol as volatile hydrogen bond donor compound (melting point 41° C.) were mixed in benzamide:metaxalone:phenol molar ratio of 1:1:10. The melting point of the mixture after standing was found to be below 0° C. X-ray diffraction results of the product are shown in FIG. 18.

### Example 21: Co-Crystalline Metaxalone/Carbamazepine

In example 21, a mixture was formed of metaxalone/carbamazepine/phenol in a carbamazepine:metaxalone:phenol molar ratio of 1:1:10. The melting point of the mixture after standing was found to be below 0° C. X-ray diffraction results of the product are shown in FIG. 19.

### Example 22: Co-Crystallization Urea/4-Nitrophenol

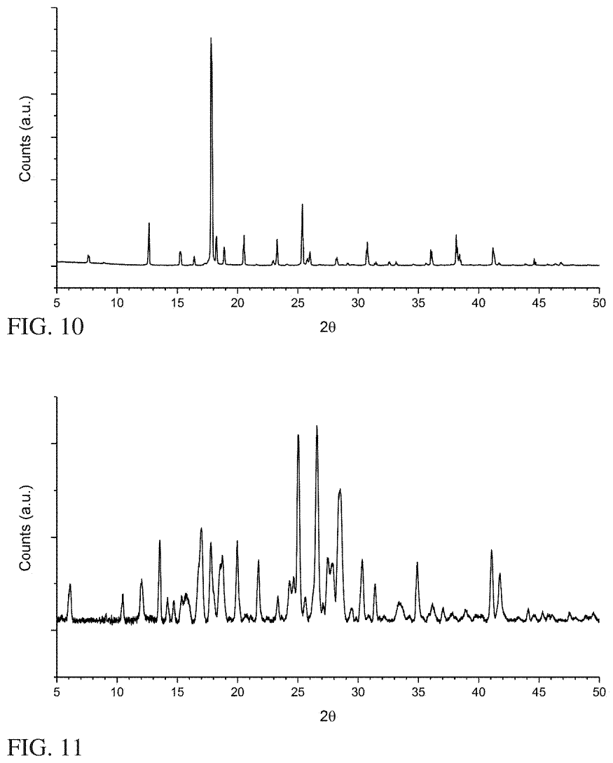

In Example 22, a mixture was formed of urea/4-nitrophenol/phenol in a urea:4-nitrophenol:phenol molar ratio of 1:1:10. The melting point of the mixture after standing was found to be below 0° C. An X-ray diffraction powder pattern for the resulting crystalline solid is shown in FIG. 10.

### Example 23: Co-Crystallization p-Coumaric Acid/Nicotinamide

In Example 23, a mixture was formed of p-coumaric acid/nicotinamide/phenol in p-coumaric acid:nicotinamide:phenol molar ratio of 1:1:10. The melting point of the mixture after standing was found to be below 0° C. An X-ray diffraction powder pattern for the resulting crystalline solid is shown in FIG. 11.

### Example 24: Co-Crystallization of 4-Hydroxybenzoic Acid/Tebuconazole

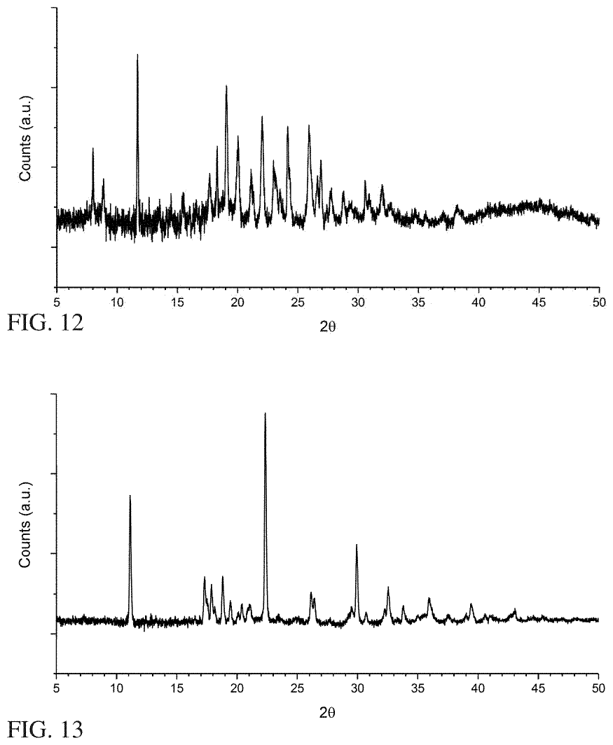

In Example 24, a mixture was formed of 4-hydroxybenzoic acid/tebuconazole/phenol in 4-hydroxybenzoic acid:tebuconazole:phenol molar ratio of 1:1:10. The melting point of the mixture after standing was found to be below 0° C. An X-ray diffraction powder pattern for the resulting crystalline solid is shown in FIG. 12.

### Example 25: Co-Crystals of 2′-Aminoacetanilide and Tetracyanoquinodimethane (TCNQ)

2′-Aminoacetanilide, TCNQ and phenol were mixed in the ratio 1:1:3 to form a stable liquid. The phenol was left to leave the system, generating cocrystals of 2′-Aminoacetanilide and TCNQ. The results of powder X-ray diffraction of the product are shown in FIG. 21.

### Example 26: Co-Crystals of Theobromine and Vanillic Acid

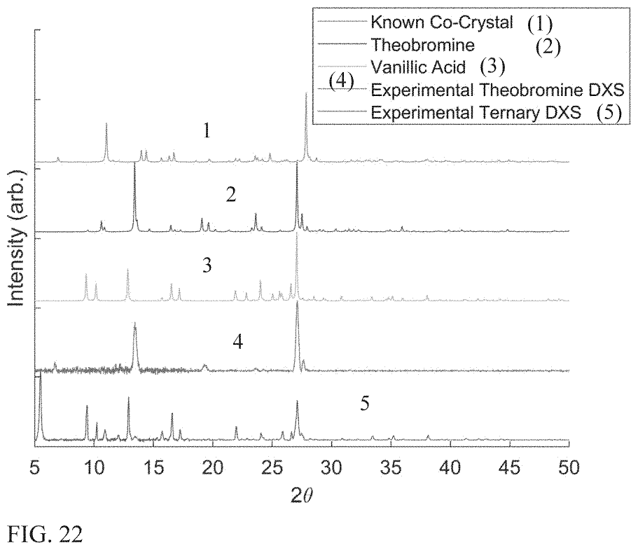

Theobromine, vanillic acid and phenol were mixed in the ratio 1:1:20, to form a stable liquid. The phenol was left to leave the system, generating cocrystals of theobromine and vanillic acid. X-ray diffraction results are shown in FIG. 22.

In each Example, the liquid mixture was exposed to the atmosphere, allowing phenol to evaporate, and resulting in formation of cocrystals of the other two organic compounds present in the system. The cocrystals are either present as fully crystalline materials, or as a homogeneous amorphous glass.

In each Example, the formation of cocrystals of the two organic compounds in each system were confirmed through powder X-Ray diffraction.

1-23. (canceled)

24. A solidification or crystallization method, the method comprising:
forming a mixture of a plurality of components including at least a first organic compound and at least one volatile co-former organic compound, wherein either the first organic compound or the volatile co-former organic compound comprises a hydrogen acceptor moiety and the other comprises a hydrogen donor moiety, such that hydrogen bonds between the first organic compound and the volatile co-former organic compound are allowed to form;
allowing the mixture to stand for a sufficient time for the mixture to liquify at a temperature below that of the melting points of the components, such that a liquid mixture is formed from the mixture; and
allowing the volatile co-former organic compound to evaporate, resulting in crystallization of at least the first organic compound.

25. The method of claim 24, wherein the mixture of the plurality of components further includes a second organic compound, and
wherein either: A) the volatile co-former organic compound comprises a hydrogen acceptor moiety and the first organic compound and the second organic compound each comprise a respective hydrogen donor moiety, or B) the volatile co-former organic compound comprises a hydrogen donor moiety and the first organic compound and the second organic compound each comprise a respective hydrogen acceptor moiety,
such that forming the mixture allows the formation of hydrogen bonds between the first organic compound and the volatile co-former organic compound and between the second organic compound and the volatile co-former organic compound, such that allowing the volatile co-former organic compound to evaporate results in co-crystallization of at least the first and second organic compounds.

26. The method of claim 24, wherein allowing the mixture to stand comprises sealing the mixture in a vessel and allowing the mixture to stand in the vessel.

27. The method of claim 24, wherein the sufficient time is a time period of 8 hours to 10 days.

28. The method of claim 24, wherein allowing the mixture to stand for the sufficient time further comprises heating the mixture.

29. The method of claim 25, wherein the first organic compound and the second organic compound each comprise a respective hydrogen bond acceptor moiety and the volatile co-former organic compound comprises a hydrogen bond donor moiety.

30. The method of claim 29, wherein the first organic compound, the second organic compound, or each of the first and second organic compounds independently contains a functional group selected from the group consisting of amide, acid, alcohol, ketone, aldehyde, amine, ester and halogen.

31. The method of claim 25, wherein the first and second organic compounds each comprise a respective hydrogen bond donor moiety and the volatile co-former organic compound comprises a hydrogen bond acceptor moiety.

32. The method of claim 25, wherein a molar ratio of the volatile co-former organic compound and either the first organic compound or the second organic compound is in the range 20:1 to 1:1.

33. The method of claim 25, wherein a molar ratio of the first organic compound and the second organic compound is in the range 1:5 to 5:1.

34. The method of claim 25, wherein the hydrogen bond acceptor or donor moiety of the first organic compound and the second organic compound is not so sterically hindered that it is unable to interact with the hydrogen bond donor or acceptor moiety of the volatile co-former compound.

35. The method of claim 24, wherein the volatile co-former organic compound has a boiling point of 289° C. or lower.

36. The method of claim 24, wherein the volatile co-former organic compound is selected from the group consisting of cyclohexanol and a hydroxy-functionalised aromatic compound.

37. The method of claim 36, wherein the volatile co-former organic compound is a hydroxy-functionalized aromatic compound of formula: 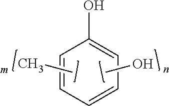
wherein, independently, n is 0 or 1 and m is 0,1, or 2.

38. The method of claim 24, wherein the volatile co-former organic compound is selected either from the group consisting of phenol, hydroquinone, resorcinol, catechol, a cresol, a xylenol, and cyclohexanol, or from a mixture of two or more of phenol, hydroquinone, resorcinol, catechol, a cresol, a xylenol or cyclohexanol.

39. A eutectic mixture comprising phenol and a first organic compound selected either from the group consisting of carbamazepine, paracetamol, metacetamol, ibuprofen, tadalafil, metaxalone, benzamide, 2-methoxybenzamide, 2-ethoxybenzamide, indomethacin, lamotrigine, and harmine, or from a mixture of two or more of carbamazepine, paracetamol, metacetamol, ibuprofen, tadalafil, metaxalone, benzamide, 2-methoxybenzamide, 2-ethoxybenzamide, indomethacin, lamotrigine, and harmine.

40. The eutectic mixture of claim 39, wherein the eutectic mixture is liquid at room temperature and pressure.

41. The eutectic mixture of claim 39 comprising benzamide, metaxalone, and phenol, wherein the eutectic mixture is liquid at room temperature and pressure.

42. The eutectic mixture of claim 39 comprising metaxalone, carbamazepine, and phenol, wherein the eutectic mixture is liquid at room temperature and pressure.

43. A co-crystalline solid comprising metaxalone and a second component comprising at least one of benzamide or carbamazepine; wherein a molecular ratio of metaxalone:the second component is in the range 2:1 to 1:2.
